(19) United States
(12) Patent Application Publication
(10) Pub. No.: US 2020/0110038 A1
(43) Pub. Date: Apr. 9, 2020

(54) METHOD AND APPARATUS FOR MONITORING AIR SAMPLES FOR ILLICIT DRUGS

(71) Applicant: RJ LEE GROUP, INC., MONROEVILLE, PA (US)

(72) Inventors: ANTONIOS TASOGLOU, PITTSBURGH, PA (US); JAMES B. DUNCAN, KENNEWICK, WA (US); HEINZ J. HUBER, NOVELTY, OH (US)

(73) Assignee: RJ LEE GROUP, INC., MONROEVILLE, PA (US)

(21) Appl. No.: 16/588,085

(22) Filed: Sep. 30, 2019

Related U.S. Application Data
(60) Provisional application No. 62/743,207, filed on Oct. 9, 2018.

(51) Int. Cl. G01N 21/78; G01N 33/497; G01N 33/00; G01N 31/22

(52) U.S. Cl. CPC G01N 21/783; G01N 31/224; G01N 33/0059; G01N 33/497

(57) ABSTRACT

A colorimetric sensor for drugs and an associated method provide for delivery of particles to the sensor and a colorimetric reagent containing element having a plurality of dyes structured to provide a color change when the airborne particles indicate the presence of fentanyl or fentanyl analogues. The color change may be monitored by an optical identifier which delivers responsive information to a programmable controller which, in turn, may activate a visual alarm or an audible alarm.

### CROSS REFERENCE TO RELATED APPLICATION

The present application claims the benefit of provisional application Ser. No. 62/743,207 which is incorporated herein.

### BACKGROUND OF THE INVENTION

### 1. Field of the Invention

The present invention discloses a method and apparatus for monitoring air samples for the presence of illicit drugs such as fentanyl and fentanyl analogues and, more specifically relates to employing separation of specific particle size ranges within an ambient air stream and exposing the particulate matter to a dye.

### 2. Description of the Prior Art

The United States Drug Enforcement Administration (USDEA) has issued a nationwide alert about the dangers of fentanyl and fentanyl analogues. Fentanyl is a schedule II narcotic under the Controlled Substances Act and known as a highly effective and fast analgesic and anesthetic drug for over 50 years. It can be delivered to the human body through a variety of ways primarily through oral intake, inhalation or injection. Fentanyl is highly lipid soluble and rapidly crosses the blood-brain barrier which makes it a highly potent analgesic, about 100 times more potent than morphine and 50 times more than heroin. Due to its rapid onset and short duration in effects it is still considered one of the best treatments for the pain management in cancer patients when other opioid medications are no longer effective. Licit tradenames are, for example, Actiq, Fentora, Lazanda or Duragesic.

Fentanyl abuse is not a recent problem, it initially appeared in the mid-1970s but only since the 1990s the public awareness has been rising on the deadly aspects of this drug. Darke, S. et al. (1999). Fluctuations in heroin purity and the incidence of fatal heroin overdose. Drug and Alcohol Dependence 54, 155-161. After a brief rise in 2006, it wasn't until 2013 that a rapid increase in positive identification of fentanyl and related substances in drugs analyzed for law enforcement has been reported (see numbers in National Forensic Laboratory Information System). This in turn has caused a significant increase in the threat to health of first responders and public safety in general. Since then, programs have expanded substantially in the United States and Canada to become a leading public health intervention for the prevention of overdose mortality. Oluwajenyo Banjo, M. et al. (2014). A quantitative and qualitative evaluation of the British Columbia Take Home Naloxone program. CMAJ Open, 2(3), E153-E161. http//dx.doi.org/10.9778/cmajo.20140008.

In parallel, the amount of fentanyl involved deaths by overdose was at less than 1 per 100,000 people in the years until 2013 and has significantly increased to 3 in 100,000 in 2015, to more than 6 in 100,000 in 2016. Within the US, there is a split in regional variation, with 75% of cases identified in the Northeast or Midwest region. In January 2018, Pennsylvania was the latest state to declare the opioid epidemic a public health emergency which allows amongst other aspects to leave the overdose-reversing drug naloxone with emergency responders at all times.

Fentanyl detection in the field is a non-trivial task. Most of the times the substance is mixed with either heroin or cocaine or other drugs. The amount of actual fentanyl is usually within a small percent range; however, in recent times nearly pure fentanyl samples were found and analyzed.

Current methods for the detection of fentanyl include highly sophisticated laboratory techniques like HPLC-MS, GC-MS or FTIR. Two other techniques were recently validated upon their usefulness for in-field analysis of mixtures of fentanyl and fentanyl analogues: Sisco, E. et al. (2017) Rapid detection of fentanyl, fentanyl analogues, and opioids for on-site or laboratory based drug seizure screening using thermal desorption DART-MS and ion mobility spectrometry. Forensic Chemistry 4, 108-115. Ion-mobility spectrometry (IMS) and thermal desorption direct analysis in real time mass spectrometry (TD-DART-MS). It resulted that both could identify fentanyl in mixtures that contained less than 1% of fentanyl and one of them, ion mobility spectrometry has the potential to be used in the field, as a handheld device. The other, TD-DART-MS is more sensitive, but, due to its size and price are primarily of use in forensic laboratories. The main advantage of both instruments is that a swab of the outside can be used and the bags do not have to be opened.

In addition, several analytical devices are on the market that use the interaction of a light beam with the material. For example, laser based instruments can be used to identify fentanyl within other opioids even when the material is packaged in plastic without the need to unwrap. This is also true for options that are based on Raman like the TruNarc™ or TacticID® analyzers. These devices work very well for the day-to-day in-field testing to scan pills, powders and other substances; however, these tests are legally considered “presumptive” tests which requires further analyses in an accredited laboratory.

None of these devices analyze the air that the officer's breath or works continuously in (near) real time. The main transfer of fentanyl into the body is either by direct contact or by accidental inhalation. Direct contact has been greatly removed due to the use of the above mentioned handheld devices, however, the amount of opioids in the air can be large enough to cause respiratory distress or worse.

### SUMMARY OF THE INVENTION

The present invention involves a colorimetric analytical device and associated method for detecting the presence of illicit drugs such as fentanyl in air. The system may be operated on a continuous basis and provide real time results. The system is premised on active sampling of ambient air which may be indoor and outdoor.

The sampled air undergoes an internal preselection based on particle size of particulate matter using miniaturized impactor technology or cyclones. The preselected air stream with particulate matter of specific size is then transferred and exposed to a liquid chemical reagent that offers unique chemical reactions with fentanyl or fentanyl analogues.

The method of providing the chemical reagent is identified to be either through a pre-prepared film in the form of such as a cassette, or prepared in situ by the use of a set of rollers or a miniature conveyor belt, for example.

The presence of fentanyl or equivalent target analytes results in a predetermined color change while is recorded and automatically evaluated. Audible and optical alarms are on the device as well as an alarm signal is transferred to an external module.

In its preferred embodiment, the device provides alarms for the presence of fentanyl and fentanyl analogues such as carfentanyl, for example. In another embodiment, the device provides alarms for the presence of additional synthetic drugs, such as synthetic cannabinoids, for example.

It is an object of the present invention to provide an efficient method and apparatus for monitoring of air samples for the presence of illicit drugs such as fentanyl and fentanyl analogues.

It is a further object of the present invention to provide such a system which may be operated in a continuous manner.

It is a further object of the present invention to provide such a system which may be operated on a real time basis.

It is a further object of the present invention to employ a colorimetric sensor and related method in a portable drug sensing system.

It is another object of the present invention to provide a compact, portable self-contained sensor for fentanyl and fentanyl adjuvants.

These and other objects of the invention will be readily apparent to those skilled in the art from the following detailed description and the appended claims.

### BRIEF DESCRIPTION OF THE DRAWINGS

FIG. 1(a) is a molecular structure of Heroin.

FIG. 1(b) is an illustration of the molecular structure of Fentanyl.

FIG. 2 is a schematic perspective view of an air flow sensor of the present invention.

FIG. 3 is a schematic illustration of a camera embodiment of the air flow sensor.

FIG. 4 is a schematic illustration of a conveyer belt embodiment of the present invention.

FIG. 5 is a schematic illustration of a portion of the sensor of FIG. 4.

FIG. 6 is an embodiment of the invention employing a camera.

FIG. 7 is a conceptual illustration of the camera showing dye impregnation within the sensor.

FIG. 8 is an embodiment which employs the camera and a conveyer.

### DESCRIPTION OF THE PREFERRED EMBODIMENTS

The device is based on sampling ambient air indoors and outdoors, separating specific particle sizes that fentanyl and its analogues are of specific danger, sampling these particles and identifying these particles by using a colorimetric approach. The colorimetric approach of this invention has been proven to effectively work with fentanyl. The system facilitates continuous monitoring and evaluation on a real time basis

The apparatus and process of the present invention employs a sampling obtained from ambient air in determining particle size.

Fentanyl has an estimated vapor pressure of 4.6±2.7·10⁶ Pa at 25° C. and is expected to exist solely in the particulate phase in the ambient atmosphere. Lyman W J; p. 31 in Environmental exposure from chemicals Vol I, Neely W B, Blau G E, eds, Boca Raton, Fla.: CRC Press (1985); and Gupta, P. K. et al. (2008) Vapor Pressure and Enthalpy of Vaporization of Fentanyl. J. Chem. Eng. Data 53, 841-845. Fentanyl and analogues are water soluble, so expedient decontamination (rinsing) of any contacted areas with water is advisable. Fentanyl in its hydrochloride form (the most common street form) is more soluble than the citrate form.

According to the InterAgency Board. Recommendations on selection and use of personal protective equipment and decontamination products for first responders against exposure hazards to synthetic opioids, including fentanyl and fentanyl analogues, 2017, the particle size of synthetic opioid powders typically ranges from 0.2 to 2.0 microns, and the powders are easily aerosolized, presenting primarily a respiratory hazard.

The mechanisms of deposition of aerosols on the human body depend on fundamental aerosol kinetics. Several mechanisms account for the deposition of aerosols within the respiratory tract. For particles with a diameter range from 1 to 10 μm, the two govern mechanisms are inertial impaction and gravitational sedimentation while a third mechanism, Brownian diffusion, is important for aerosols <1 μm diameter. Depending on their particle size, inhaled drug particles will deposit in different regions of the lung causing different effects. Particles <1 μm are likely to reach the peripheral airways and alveoli or be exhaled, particles 1-5 μm will deposit in the large and conducting airways, while particles >5 μm will predominately deposit in the oropharynx. Research has been published in Newman, S. P. (1985) Aerosol deposition considerations in inhalation therapy, Chest 88(Suppl. 2), S152S-S160; and Everard, M. L. (2001) Guidelines for devices and choices. J. Aerosol Med. 14(Suppl. 1), S59-S64.

The separation of the particles inside the device will occur based on cascade impactor technology. Cascade impactors are low cost and robust instruments and have been widely used in aerosol instrumentation to separate aerosols in an artificially generated jet stream according to their aerodynamic diameter. Particle size separation is carried out by the inertial impaction of the particles. In a typical Cascade impactor, the particles are separated in three stages. In the first stage, the particles with a diameter from 2.5 to 10 μm are collected in the first impaction plate. Similarly, in the second stage particles from 1 to 2.5 μm are collected in the second impaction plate and finally particles smaller than 1 μm are collected in the third impaction plate or on a filter. In our device, the intake will contain a mesh filter in order to remove large particle such as dust and grime. A double set of impactor plates is the conservative approach to remove the particles that are less harmful to the body. An alternative embodiment would use cyclones to separate the particulate matter.

Colorimetry is advantageous compared to other instrumental techniques for low cost in-field analysis, because the analytical signal (i.e., color) can be easily detected using a optical identifier, e.g., a camera. Many commercial available colorimetric sensors exist for various analytes of interest (i.e., pollutants, industrial chemicals, bio-compounds etc.). Even though not so common, colorimetric sensing elements have been also used for the detection of compounds in air; currently a portable detection of formaldehyde in air is available where formaldehyde present in air causes a color change to a chemically impregnated tablet

The device incorporates a thin and flexible colorimetric sensing element that is loaded with more than five different colorimetric reagents; different reagents are specially separated to avoid cross contamination. Upon reaction with air particles of fentanyl the color of the sensing element changes; an optical identifier (e.g., miniaturized camera) records the image of a sensing area and software analyzes the images and exacts the concentration of fentanyl in air (in mg/L).

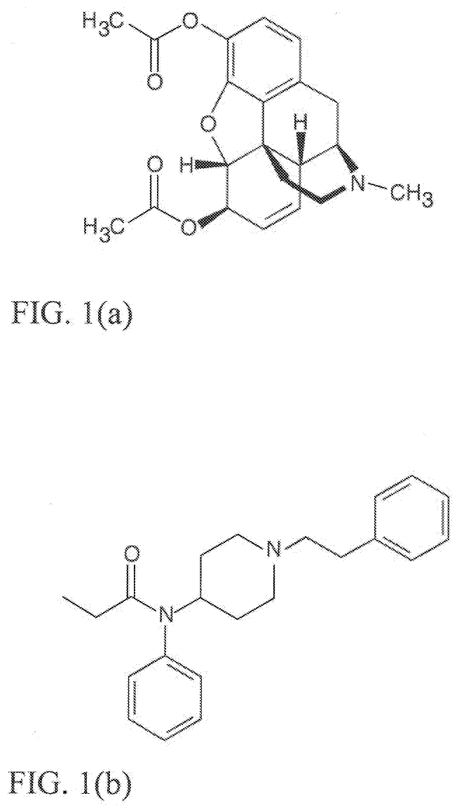

The colorimetric sensing elements can allow the selective detection of fentanyl and its analogues even when other narcotics, such as heroin and cocaine, for example and compounds, mannitol, lactose, dipyrone, baking soda coexist even in 100-times higher concentrations for two reasons: i) Fentanyl gives color products with a number of colorimetric reagents and ii) Fentanyl and its analogues have significantly different chemical structures compared to other narcotics such as heroin and cocaine. FIG. 1 (a) illustrates the molecular structure of heroin. FIG. 1(b) illustrates the molecular structure of fentanyl. The reaction with different reagents causes different colors.

The dyes that are more sensitive to fentanyl signals the presence of fentanyl in the air and the dyes more sensitive to interference allow for the correction of the analytical signal from interferences. Meaningful estimation of the true concentration of fentanyl in the sampled air. In general, the color of a sensing element is prone to be influenced by solid airborne particles and other compounds (i.e., interferants) that can co-react with the analyte of interest, and result to false positive or false negative results. To eliminate false negative and false positive results, a number of dyes (N≥5) is used on the sensing elements that react selectively with both fentanyl or possible interferants. The dyes that is more selective to fentanyl signals the presence of fentanyl in air, and the dyes more selective to interferants allow for the correction of the analytical signal from interferences and estimation of the true concentration of fentanyl in air.

Previous research studies have shown that several colorimetric reagents such as the Marquis Reagent, Mandelin Reagent, or Liebermann Reagent and dyes such as azo dyes or xanthene dyes can be used to detect fentanyl and other narcotics. For example, Marquis Reagent reacts with fentanyl citrate and the color is changed from orange to brown while Mandelin Reagent reacts with fentanyl and turns green. Several azo dyes also react with fentanyl and give color products; a comprehensive summary of responses to fentanyl and analogues with azo dyes concluded that Acid Orange 8 or Acid Red 88 are the most promising dyes for this analysis. Jelinkova et al. (2013) The possibility of identifying selected opioids by spectral analysis. Mil. Med. Sci. Lett. 84, 172-179. A recent publication has suggested a different approach based on Eosin (Eosin-Y). Kangas M. J., Symonsbergen D. J., and Holmes A. E. (2017) A new possible alternative colorimetric drug detection test for fentanyl. Organic and Medicinal Chem IJ 4(4): 555645. The same research group introduced the DETECHEM® colorimetric array in 2010 as a functional instrument to detect multiple drugs within mixtures of substances. Burks, R. M., Pacquette S. E., Guericke, M. A., Wilson, M. V., Symonsbergen, D. J., Lucas, K. A., Holmes A. E. (2010) DETECHIP®: A Sensor for Drugs of Abuse. J. Forensic Sci. 55, 723-727.

The colorimetric approach for detecting fentanyl in air also has the benefit of being easily adjusted to new compounds. One example of a current shift is the significant rise of Carfentanil in the US in 2016. This drug is normally used as a large animal tranquilizer and is 100 times more potent than fentanyl implying a serious reduction in fatal dose. In addition to Carfentanil, Acetyl fentanyl, Furanyl fentanyl and 3-methylfentanyl were detected in the 100s to 1000s of cases in 2016 (source DEA). In addition, recent developments indicate a tendency to mix fentanyl with synthetic cannabinoids and other chemical substances for enhanced effects of drug abuse.

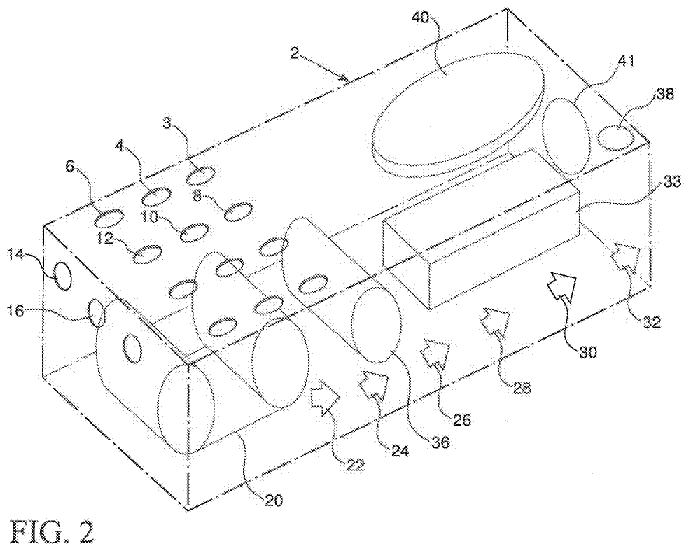

The compact self-contained sensor design is illustrated in FIG. 2. A housing 2 has a plurality of air sampling openings such as 3, 4, 6, 8, 10, 12, 14, 16, for example. The openings 3, 4, 6, 8, 10, 12, 14, 16 are covered with a mesh filter material (not shown) to resist entry of larger particles into the housing interior. The air is then directed towards the detection unit 20. By varying the widths of the air flow passage, an air stream of high velocity is generated. This air stream drives certain particles to remain in the center and hit the target dyes which will be described hereinafter while the air flow is around the sensing element and exits at the opposite side of the device as indicated by arrows 22, 24, 26, 28, 30 and 32. The air flow is preferably driven by a micro turbine 36 to allow the flow rate to be provided in liters/minutes range. The exact flow rate is determined by factors such as maximum flow possible, minimum flow needed to generate the jet stream and optimized flow to separate the fractional particles with the highest probability of being fentanyl.

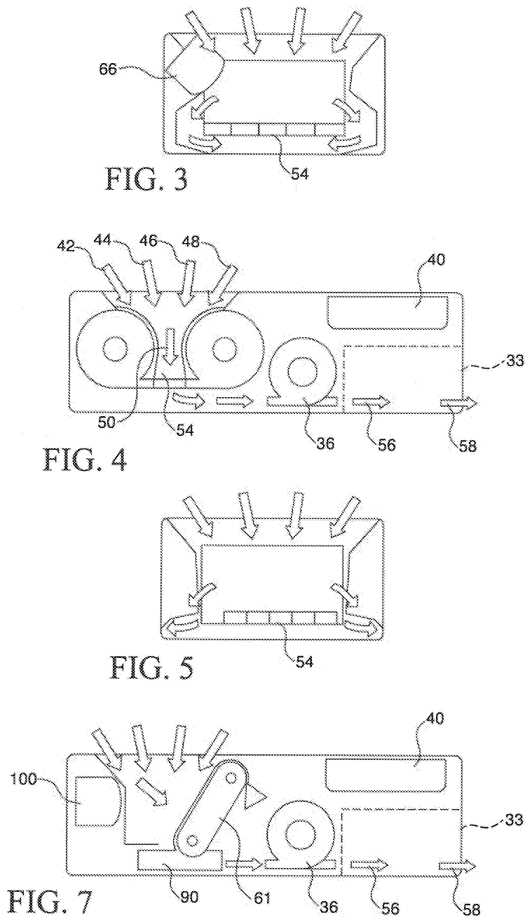

The dyes may be introduced to the air stream by one of two optional methods. One approach is to employ the concept of a film roll with the dyes printed thereon. Another concept is of a conveyor belt that dips into the dyes. The former is illustrated in FIGS. 2 through 6 and the latter approach is illustrated in FIGS. 7 and 8 which will be discussed hereinafter. The button battery 40 serves to energize the system including the underlying electronic containing programmable controller 33. The sensor is preferably also provided with an audible alarm 38 and a visual alarm 40 which can be calibrated to be activated under the control of programmable controller 33. As shown in FIG. 4, the air as exemplified by arrows 42, 44, 46 and 48 enters the housing and as shown by arrow 50, impinges upon the film 54 with the air exiting in the direction shown by arrows 56 and 58. In FIGS. 3 and 5 the arrows indicate the direction of air flow. In FIG. 3, the camera 66 is also shown.

The detection design is preferably based on an exposure time of up to three minutes. This means that any part of the filter that is visible to the camera will be exposed to the air stream for up to three minutes and then moved away. The actual minimal duration is driven by the detection limit of the device; the maximal duration is driven by the general understanding that an exposure of three minutes to an overdose of fentanyl in air can cause irreparable damages in living bodies. This replacement of the active zone is necessary to prevent the system from degrading in detection limit over time by accumulating non-target particles on the surface. The creation of a filter layer would passivate the system and cause false negative responses.

Referring again to FIG. 2, the system is powered by a battery 40, an audible alarm speaker 41 and a visual alarm 38, all of which are integrated in the housing 2. The device also has the ability to communicate with external devices to provide an alarm. The electronics controller 33 contains the control parts for the motor, a general on/off switch and the control over the readout of the camera. They include an “analytical” element that can take the images from the optical identifying part, e.g., a camera, analyze them, perform chemometric analyses and send the signal to the alarm. The alarms will be set differently for warning about fentanyl or analogues and for malfunction of the sensor such as no airflow identified by the integrated flow meter or lack of dyes.

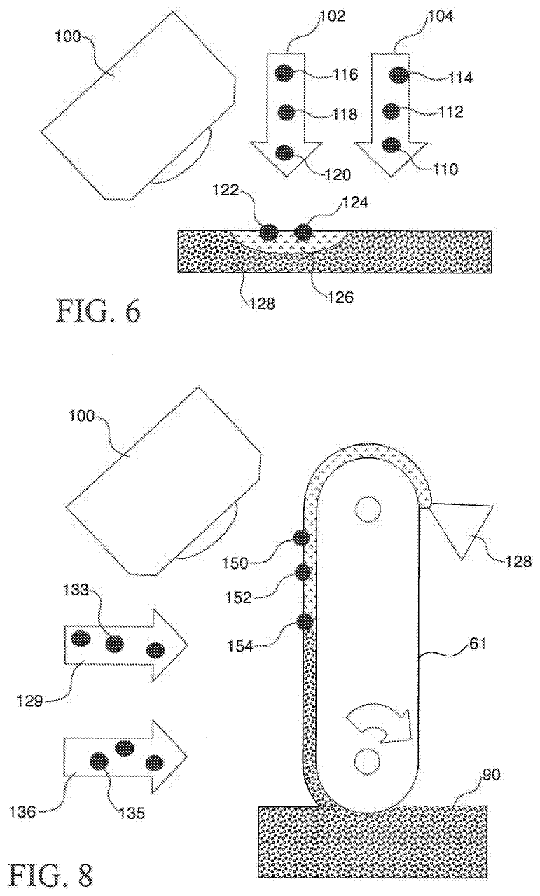

Referring to FIG. 6, a conceptual representation of the detection unit is provided. It shows the setup of the camera 100 on the left side while the arrows 102, 104 represent air pockets with the fentanyl particles being represented, for example, by reference numbers 110, 112, 114, 116, 118, 120. When fentanyl particles hit the dye, a chemical reaction takes place that changes the color of the dyes 122, 124 on dye reservoir 126 which is loaded on colorimetric reagent element 128. The film gets moved within three minutes to provide fresh dye for the next measurement cycle. This move can either be a continuous slow drag or be performed incrementally.

Referring to FIGS. 7 and 8, a dye reservoir 90 where a conveyer belt 61 or set of rollers (not shown) dip into and pull a film of the dyes onto it. The air flow is indicated by arrows 129, 131 with the black circle such as 133, 135, indicating fentanyl which will impinge on belt 61. When exposed to air containing fentanyl, the dye will change color. After three minutes, the dye gets removed by either physical, electrochemical or other means as indicated by the triangle 128 in FIG. 8, and the clean roll dips again into the dye again. The benefit of this setup is the lack of need for pre-printed film; the drawback is a need to contain the dye reservoir, keep the individual dyes from mixing and collect the removed dried dye.

The preferred embodiment has overall dimensions of a width of 1-1.5 inches, a length of 1.5-2.5 inches and a height of 0.35-.75 inches. The main driver for the overall dimensions is the minimum size of filter exposed to the airflow to obtain the required detection limit. If desired, the dye containing part may be designed as a removable cartridge that can be exchanged with the rest of the sensor remaining. This significantly increases the lifetime of the sensor, therefore, reducing overall costs of operation and increasing acceptance by the personnel. The cartridges are designed to perform over a duration of about 12-48 hours of continuous monitoring.

Personnel deployment: As its primary operational usage, the sensor can be worn on a wrist. Since the arms will be usually in front of the person while performing the job, such as to open doors, these parts will be the first to get exposed to fentanyl bearing air; specifically, they will be exposed before the person breaths it in. The sensor is being mounted on a wristband and is able to snap off. This allows for a person when being suspicious of a rooms contents to leave the sensor behind, leave the premise and wait for the three minutes whether or not an alarm is sent to their remote devices. Other options are to wear the sensor at the front of the body with the intake facing upwards or on the shoulder facing forward so that the air is sampled from the breathing zone.

Another mode of operation is for canine deployment units to mount the sensor on the collar (for larger dogs) or on a harness (for smaller dogs). Since heroin and fentanyl usually appear in intermixed form, a canine will respond to the heroin and give signal. By knowing whether or not the dog is also exposed to fentanyl allows the handler to react appropriately in a situation of exposure.

Whereas particular embodiments of the invention have been described for purposes of illustration, it will be evident to those skilled in the art that numerous variations of the details of the present invention may be made departing from the invention as defined in the appended claims.

What is claimed is:

1. A colorimetric sensor for drugs comprising
a housing,
a plurality of openings in said housing for receiving airborne particles,
a colorimetric reagent containing element for receiving said particles,
said colorimetric reagent containing element surface having a plurality of colorimetric reagent structured to provide a color change when particles contain fentanyl or fentanyl analogues contact them.

2. The colorimetric sensor of claim 1 including
said colorimetric reagent element containing at least five said reagents.

3. The colorimetric sensor of claim 1 including
an optical identifier for sensing said color change,
said sensor having a programmable controller for receipt of color information, and
an optical identifier for sensing said color change and responsively delivering said color identifying information to said program controller.

4. The colorimetric sensor of claim 3 including
said sensor having a visual alarm and an audible alarm,
said programmable controller responsive to receipt of color information indicating the presence of fentanyl or fentanyl analogues above a predetermined level emitting an alarm signal to at least one of said visual alarm and said audible alarm.

5. The colorimetric sensor of claim 1 including
said colorimetric reagent containing element having dyes printed thereon.

6. The colorimetric sensor of claim 2 including
said colorimetric reagent containing element being established through a conveyer belt that dips into a reservoir of said colorimetric reagent.

7. The colorimetric sensor of claim 1 including
said sensor having an air pump driven by a suitable motor for evacuating air after it has come in contact with said colorimetric reagent containing element.

8. The colorimetric sensor of claim 1 including
said sensor housing being miniaturized so as to be wearable on the arm of a human being.

9. The colorimetric sensor of claim 1 including
said sensor housing having an external width dimension of about 1 to 1.5 inches, length of about 1.5 to 2.5 inches and a height of about 0.35 to 0.75 inches.

10. The colorimetric sensor of claim 1 including
said sensor housing being miniaturized so as to the mountable on a collar or harness for a canine.

11. The apparatus of claim 1 including
said sensor having a battery for energizing said sensor.

12. The apparatus of claim 1 including
said sensor having a pump motor for establishing air flow through said sensor.

13. A method of colorimetric sensing for drugs comprising
providing a housing having a plurality of openings in said housing for receiving airborne particles,
a colorimetric reagent containing element for receiving said particles,
introducing airborne particles into said housing,
said colorimetric reagent containing element having a plurality of colorimetric reagents structured to provide a color change when said particles contain fentanyl or fentanyl analogues, and
monitoring by employing an optical identifier, the colorimetric reagent containing element for color changes.

14. The method of claim 13 including
providing said sensor with electronic programmable controller for receipt of color information from said optical identifier and delivering said color information from said optical identifier to said electronic programmable controller.

15. The method of claim 13 including
providing said sensor with a visual alarm and an audio alarm, and
responsive to receipt of said color information by said programmable controller indicating the presence of fentanyl or a fentanyl analogue, and
emitting an alarm signal through at least one of said visual alarm and said audio alarm.

16. The method of claim 13 including
said colorimetric reagent containing element having a film having dyes printed thereon.

17. The method of claim 13 including
said colorimetric reagent containing element being established through a conveyer belt that dips into a reservoir of said colorimetric reagent.

18. The method of claim 13 including
said colorimetric reagent containing element having at least five different reagents secured thereto.

19. The method of claim 13 including
moving said airborne particles through said sensor under the influence of an air pump in order to evacuate air after it has come in contact with said colorimetric reagents.

20. The method of claim 13 including
said sensor being miniaturized and wearable on the arm of a human being.

21. The method of claim 20 including
providing said sensor having an external width dimension of about 1 to 1.5 inches, length of about 1.5 to 2.5 inches and a height of about 0.35 to 0.75 inches.

22. The method of claim 13 including
miniaturizing said sensor housing so as to be mountable on a collar or harness of a canine.

23. The method of claim 13 including
operating said method on a continuous basis.

24. The method of claim 13 including
operating said method of a real-time basis.

25. The method of claim 13 including
prior to initiating the colorimetric sensor procedure, filtering said airborne particles to eliminate portions thereof.
